(19) United States
(12) Patent Application Publication
(10) Pub. No.: US 2024/0081406 A1
(43) Pub. Date: Mar. 14, 2024

(54) NON-COMBUSTIBLE AEROSOL PROVISION DEVICE AND SYSTEM

(71) Applicant: NICOVENTURES TRADING LIMITED, London (GB)

(72) Inventors: Jeremy CAMPBELL, London (GB); Matthew WRIGHT, Cambridge Cambridgeshire (GB); Daniel Anthony WALTON, Cambridge Cambridgeshire (GB); Andy FINCH, St. Ives Cambridgeshire (GB); Jon Peter Frederick SPRATLEY, St. Ives Cambridgeshire (GB); Daniel Thomas AHEARN, St. Ives Cambridgeshire (GB); Andrew RIchard Buchanan HALKET, London (GB); Conrad Walker HIGGS, Norfolk (GB); Hielke DE LANGE, CN Houten (NL)

(21) Appl. No.: 18/262,999

(22) PCT Filed: Feb. 1, 2022
(86) PCT No.: PCT/EP2022/052360

§ 371 Date: Jul. 26, 2023

(30) Foreign Application Priority Data
Feb. 3, 2021 (GB) 2101467.5

(51) Int. Cl. A24F 40/46; A24F 40/20; A24F 40/465; A24F 40/60; H05B 6/10

(52) U.S. Cl. CPC A24F 40/46; A24F 40/20; A24F 40/465; A24F 40/60; H05B 6/108

(57) ABSTRACT

A non-combustible aerosol provision device for generating an aerosol from an aerosol-generating material is provided. The device includes a chamber for receiving a consumable including aerosol-generating material to enable the non-combustible aerosol provision device to generate an aerosol from the aerosol-generating material. The device further includes an adjustment mechanism to allow a user to adjust a dimension of the chamber to allow the chamber to accommodate, one at a time, each of a plurality of consumables having different sizes.

### PRIORITY CLAIM

The present application is a National Phase entry of PCT Application No. PCT/EP2022/052360, filed Feb. 1, 2022, which claims priority from GB Application No. 2101467.5, filed Feb. 3, 2021, each of which is hereby fully incorporated herein by reference.

### TECHNICAL FIELD

The present disclosure relates to a non-combustible aerosol provision device and a non-combustible aerosol provision system comprising a non-combustible aerosol provision device and a consumable containing aerosol-generating material from which the non-combustible aerosol provision device is configured to generate an aerosol.

### BACKGROUND

Smoking articles such as cigarettes, cigars and the like burn tobacco during use to create tobacco smoke. Attempts have been made to provide alternatives to these articles that burn tobacco by creating products that release compounds without burning. Examples of such products are heating devices which release compounds by heating, but not burning, the material. The material may be for example tobacco or other non-tobacco products, which may or may not contain nicotine.

### SUMMARY

According to a first aspect of the present disclosure, there is provided a non-combustible aerosol provision device for generating an aerosol from an aerosol-generating material, the non-combustible aerosol provision device comprising: a chamber for receiving a consumable comprising aerosol-generating material to enable the non-combustible aerosol provision device to generate an aerosol from the aerosol-generating material; and an adjustment mechanism to allow a user to adjust a dimension of the chamber to allow the chamber to accommodate, one at a time, each of a plurality of consumables having different sizes.

The non-combustible aerosol provision device may comprise a tube, wherein an interior of the tube defines the chamber. The adjustment mechanism may allow adjustment of a dimension of the tube to thereby adjust the dimension of the chamber.

A sheet of material may be arranged to form the tube. The sheet of material may comprise two respective opposing longitudinal edges, and the adjustment mechanism may allow a position of the opposing edges of the sheet of material with respect to one another to be adjusted to adjust a diameter of the tube.

The opposing edges of the sheet of material may be configured, in at least some configurations in use, to overlap one another, and the adjustment mechanism may allow a degree of overlap of the opposing edges to be adjusted to adjust the diameter of the tube.

The opposing edges of the sheet of material may be configured such that, in at least some configurations, the opposing edges do not overlap one another, and wherein a distance between the opposing edges with respect to a circumferential direction of the tube is adjustable by the adjustment mechanism.

The tube may further comprise a second portion, wherein the second portion is configured to form a portion of the tube between the opposing edges when the opposing edges of the sheet of material are not overlapping one another.

The adjustment mechanism may comprise a frame having one or more guides and the tube may comprise one or more guide elements, each of the one or more guide elements being configured to engage with a respective one of the one or more guides to cause a or the diameter of the tube to be adjusted when the frame is moved relative to the tube.

The frame may surround the tube and the guide elements may protrude radially outwardly from the tube to engage with the guides.

The adjustment mechanism may be configured to adjust a length of the tube.

Adjustment of one of the diameter of the tube and the length of the tube may cause adjustment of the other of the diameter of the tube and the length of the tube.

A reduction in one of the length of the tube and the diameter of the tube may cause an increase in the other of the length of the tube and the diameter of the tube, and an increase in one of the length of the tube and the diameter of the tube may cause a decrease in the other of the length of the tube and the diameter of the tube.

The tube may comprise an expandable braided structure.

The tube may comprise a helical coiled structure.

The adjustment mechanism may comprise a biasing element arranged to bias the length of the tube to a maximum length and the adjustment mechanism may allow the user to work against the biasing element to decrease the length of the tube and thereby to increase the diameter of the chamber.

The adjustment mechanism may comprise a biasing element configured to act to decrease the dimension of the chamber, and the adjustment mechanism may allow the user to work against the biasing element to increase the dimension of the chamber to allow insertion of the consumable comprising aerosol-generating material.

The adjustment mechanism may allow the user to configure the chamber between a first configuration in which the chamber has a first width and a second configuration in which the chamber has a second width different from the first width.

The adjustment mechanism may comprise an actuator engageable by the user to operate the adjustment mechanism to adjust the dimension of the chamber.

The actuator may comprise a lever which is rotatable in a circumferential direction with respect to the chamber to operate the adjustment mechanism.

The actuator may be moveable in a longitudinal direction with respect to the chamber to operate the adjustment mechanism.

The actuator may allow the user to work against the or a biasing element to increase the diameter of the chamber to allow insertion of a consumable and the adjustment mechanism may be configured, when the button is released, to cause contracting of the chamber to fit the consumable.

The actuator may comprise a portion of an outer housing of the device which is moveable relative to the chamber.

The movable portion of the outer housing of the device may be a proximal portion of the housing comprising an aperture for inserting the consumable into the chamber.

The adjustment mechanism may be configured to adjust a length of the chamber.

The tube may be a susceptor element configured to be inductively heated by an inductive element of the device to thereby heat the aerosol-generating material received in the chamber.

The tube may be configured to be heated by a resistive heater to thereby heat the aerosol-generating material received in the chamber.

The device may be a tobacco heating product configured to receive and generate aerosol from each of a plurality of consumables comprising tobacco and having different sizes.

According to a second aspect of the present disclosure, there is provided a non-combustible aerosol provision device for generating an aerosol from an aerosol-generating material, the non-combustible aerosol provision device comprising: a chamber for receiving a consumable comprising aerosol-generating material to enable the non-combustible aerosol provision device to generate an aerosol from the aerosol-generating material; and an adjustment mechanism configured: to allow a user to increase a width of the chamber to allow the consumable to be inserted into the chamber; and, once the consumable is inserted into the chamber, to decrease the width of the chamber to cause the chamber to fit to a width of the consumable.

According to a third aspect of the present disclosure there is provided a non-combustible aerosol provision system comprising a non-combustible aerosol provision device according to the first aspect of the present disclosure or the second aspect of the present disclosure and at least one consumable containing aerosol-generating material, the consumable being configured to be received by the non-combustible aerosol provision device to allow the device to generate aerosol from the aerosol-generating material.

Further features and advantages of the disclosure will become apparent from the following description of various embodiments of the disclosure, given by way of example only, which is made with reference to the accompanying drawings.

### BRIEF DESCRIPTION OF THE DRAWINGS

FIG. 1 shows a schematic representation of an example non-combustible aerosol provision device according to the disclosure.

FIG. 2 shows a perspective side view of another example non-combustible aerosol provision device according to the disclosure.

FIG. 3A and FIG. 3B show top-down schematic representations of a susceptor tube of the device with the device accommodating, respectively, a first consumable and a second consumable.

FIG. 4 shows a perspective side view of an assembly including the susceptor tube and certain other components of the device of FIG. 2.

FIG. 5A and FIG. 5B show perspective cross-sectional views of the assembly comprising the susceptor tube of FIG. 4, respectively accommodating the first consumable and the second consumable.

FIG. 6 shows, in a perspective cross-sectional view, another example of a non-combustible aerosol provision device according to the disclosure.

FIG. 7 shows an assembly comprising a susceptor tube and certain other components of the device shown in FIG. 6.

FIG. 8A and FIG. 8B show in a perspective view, a portion of the assembly shown in FIG. 6 in, respectively, a first configuration and a second configuration.

FIG. 8C and FIG. 8D illustrate, in a top-down schematic view, aspects of the assembly shown in FIG. 8A and FIG. 8B in, respectively, the first configuration and the second configuration.

FIG. 9A and FIG. 9B show, each in a cross-sectional side view, another example of a non-combustible aerosol provision device according to the disclosure.

FIG. 9C and FIG. 9D illustrate, in a top-down schematic view, aspects of the device shown in FIG. 9A and FIG. 9B in, respectively, a first configuration and a second configuration.

FIG. 9E shows a perspective side view of a heating tube of the device of FIG. 9A and FIG. 9B.

FIG. 10 shows in a perspective cross-sectional view, another example of a non-combustible aerosol provision device according to the disclosure.

FIG. 11 shows a schematic perspective representation of an assembly comprising a heating tube of the device shown in FIG. 10.

FIG. 12 shows in a perspective side view, another example of a non-combustible aerosol provision device according to the disclosure.

FIG. 13A and FIG. 13B show cross-sectional side views of an assembly comprising a heating tube and other components of the device of FIG. 12.

FIG. 14A and FIG. 14B show, each in a cross-sectional side view, another example of a non-combustible aerosol provision device according to the disclosure in, respectively, a first configuration and a second configuration.

FIG. 15A and FIG. 15B show, in a side view, a helical coil defining a tube accommodating, respectively, the first consumable and the second consumable.

### DETAILED DESCRIPTION OF THE DRAWINGS

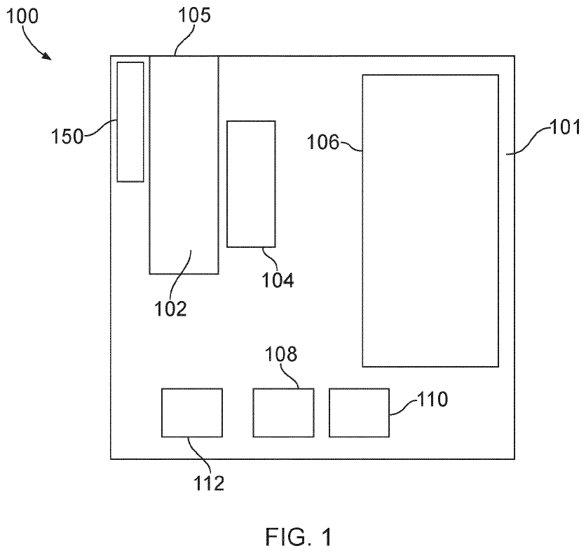

FIG. 1 is a simplified schematic view of an example non-combustible aerosol provision device 100. The non-combustible aerosol provision device 100 comprises a heating chamber 102. The heating chamber 102 is configured to receive a consumable (not shown in FIG. 1) which comprises aerosol-generating material, which may or may not comprise tobacco.

Aerosol-generating material is a material that is capable of generating aerosol, for example when heated, irradiated or energized in any other way. Aerosol-generating material may, for example, be in the form of a solid, liquid or gel which may or may not contain an active substance and/or flavorants. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material may comprise one or more active substances and/or flavors, one or more aerosol-former materials, and optionally one or more other functional material.

The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine. In some embodiments, the active substance comprises caffeine, melatonin or vitamin B12.

The one or more other functional materials may comprise one or more of pH regulators, coloring agents, preservatives, binders, fillers, stabilizers, and/or antioxidants.

The aerosol-former material may comprise one or more constituents capable of forming an aerosol. In some embodiments, the aerosol-former material may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

In this example, the non-combustible aerosol provision device 100 is for heating aerosol-generating material in a consumable to volatilize at least one component of the aerosol-generating material. The device 100 is configured to heat the aerosol-generating material in a consumable (not shown in FIG. 1) which is received in the described heating chamber 102. The device 100 comprises a heating arrangement 104 configured to provide energy for heating the aerosol-generating material in a consumable received in the heating chamber 102. In some examples, the heating arrangement 104 comprises one or more resistive heating elements arranged in thermal contact with the heating chamber 102. The flow of current against the electrical resistance of the one or more resistive heating elements generates heat. This process is called Joule, ohmic, or resistive heating.

A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms. The device that is configured to generate the varying magnetic field may also be referred to as a magnetic field generator.

In some examples, the heating arrangement 104 is an induction heating arrangement and is configured to generate a varying magnetic field in order to inductively heat a susceptor. In some examples, the susceptor is configured to define the heating chamber 102 for receipt of the consumable, as will be described below in more detail. The induction heating arrangement may comprise one or more inductors through which an alternating current is passed to generate the varying magnetic field. In some examples using induction heating, the heating arrangement 104 comprises one or more susceptors. In other examples using induction heating, the heating arrangement 104 may not comprise a susceptor and one or more susceptors may instead be provided as part of/with consumable intended for use with the device 100.

The device 100 comprises a power source 106. The power source 106 supplies electrical power to the various components of the device 100. In some examples, the power source 106 is a battery. In some examples, the power source 106 comprises a battery and a DC-DC converter, and power is supplied from the battery through the DC-DC converter. The DC-DC converter may allow the power supply 106 to supply power at a different voltage to the voltage of the battery. In some examples, the device 100 may comprise a DC to AC converter for converting a DC current from, e.g., a battery to AC current, for example, to supply power to one or more inductors of the heating arrangement 104 where the heating arrangement 104 is an induction heating arrangement. In the following examples, the power source 106 is referred to simply as the battery 106.

In the example of FIG. 1, the device 100 comprises a processor 108 in data communication with a computer readable memory 110. The processor 108 is configured to control various aspects of the operation of the device 100. The processor 108 controls the various aspects by executing instructions stored on the computer readable memory 110. For example, the processor 108 may control the operation of the heating arrangement 104. For example, the processor may control the delivery of electrical power from the battery 106 to the heating arrangement 104 by controlling various electrical components such as switches and the like (not shown in FIG. 1).

The device 100 comprises a housing 101 which forms an outer cover of the device 100 and surrounds and houses various components of the device 100. The chamber 102 in this example is configured to receive a consumable (not shown in FIG. 1) comprising aerosol-generating material from which the device 100 is configured to generate aerosol. The device 100 may generate aerosol from the consumable by the heating aerosol-generating material, for example, in the ways described above. In other examples, an aerosol may be generated from the aerosol-generating material by imparting energy to the aerosol-generating material in another way, for example, by use of ultrasonic energy.

The device 100 has an opening 105 in one end, which allows the consumable to be inserted into the chamber 102. As shown in certain examples below, in some examples a portion of the consumable may be received in the chamber 102 of the device 100, while a portion of the consumable may protrude from the opening 105 of the device 100. In examples, the portion of the consumable received in the chamber 102 comprises the aerosol-generating material from which the device 100 is configured to generate aerosol. The portion of the consumable which protrudes from the opening 105 may, for example, comprise a filter or the like, and the user may inhale the generated flow of aerosol from the portion of the consumable which protrudes from the opening 105 by inserting that portion into his or her mouth.

The device 100 may comprise a user-operable control element 112, such as a button or switch, which operates the device 100 when pressed. For example, a user may turn on the device 100 by operating the switch 112.

The device 100 may also comprise an electrical component, such as a socket/port (not shown), which can receive a cable to charge the battery 106. For example, the socket may be a charging port, such as a USB charging port. In some examples the socket may be used additionally or alternatively to transfer data between the device 100 and another device, such as a computing device. The socket may also be electrically coupled to the battery 106 via electrical tracks.

The end of the device 100 closest to the opening 105 may be known as the proximal end (or mouth end) of the device 100 because, in use, it is closest to the mouth of the user. In use, a user may insert the consumable into the opening 105, operate the user control 112 to begin heating the aerosol-generating material and draw on the proximal end of the device 100. This causes the aerosol to flow through the device 100 along a flow path towards the proximal end of the device 100. In examples, a portion of the consumable may protrude from the opening 105 and the user may draw on a proximal end of the consumable to cause the aerosol to flow to the proximal end of the device 100 to be inhaled. In other examples, the device 100 may comprise a mouthpiece on which the user inhales to draw the flow of aerosol.

An end of the device 100 opposite the proximal end and furthest away from the opening 105 may be known as the distal end of the device 100 because, in use, it is the end furthest away from the mouth of the user. As a user draws on the aerosol generated in the device 100, the aerosol flows away from the distal end of the device 100.

The device 100 may in some examples comprise a lid/cap (not shown), arranged towards the distal end of the device 100. Opening the lid/cap may provide access to the heating chamber 102. A user may, for example, open the second lid to clean components in the heating chamber 102, e.g. to remove debris from previous usage sessions.

The device 100 comprises an adjustment mechanism 150 which allows one or more dimensions of the chamber 102 to be adjusted by a user of the device 100. For example, a length and/or a width, e.g. a diameter, of the chamber 102 may be adjustable by the adjustment mechanism. Adjustment of the one or more dimensions of the heating chamber 102 in examples allows the chamber 102 to receive consumables having different sizes. For example, the adjustment mechanism may allow the user to adjust the length and/or diameter of the heating chamber 102 to provide for the dimensions of the heating chamber 102 to be suitably adjusted for accommodating an consumable having a particular size.

Accordingly, the device 100, and certain other example devices described herein, may accommodate a plurality of differently sized consumables. Further examples of devices comprising an adjustment mechanism according to the disclosure are described in more detail below. For example, the dimensions of the heating chamber may be adjustable to allow the device to receive a first consumable and the second consumable which may have different lengths and/or different diameters. In some examples, the first consumable and the second consumable are elongate consumables, which may, for example, be substantially cylindrical. The first and second consumables both comprise aerosol-generating material. The first and second consumables may, for example, comprise a distal portion containing the aerosol-generating material. The first and second consumables may also comprise a proximal portion which may comprise, for example, a filter and/or other components. Examples of the first and second consumables are described in more detail below, with reference to later figures. It should be noted that, herein, reference to consumables of different sizes or to differently-dimensioned consumables refers to consumables which are intended to be a different size to another, and does not, for example, refer to consumables which are intended to be the same size but differ in size by some small amount, due to, e.g., manufacturing tolerances. The consumables may, for example, be of different types to one another wherein each of the different types of consumables is intended to have different dimensions.

The heating chamber 102 may, in some examples, be defined by a heating tube (not shown in FIG. 1). The heating tube may surround the heating chamber 102 such that an interior hollow of the heating tube defines the heating chamber 102. The heating tube may be configured to be heated and to thereby heat a consumable received in the heating chamber 102. For example, the heating arrangement 104 may comprise one or more inductive elements and the heating tube may comprise a susceptor material and be configured to be inductively heated by the one or more inductive elements. In other examples, the heating tube may be configured to be heated by one or more resistive elements. For example, the heater tube may be configured to be formed of or in contact with a resistive heater, such as a thin film heater. Examples of such arrangements are discussed below in more detail. In other examples, the heating tube may itself not be configured to heat the consumable but may contain the consumable while the consumable is being heated. For example, the consumable may comprise susceptor material to be heated by one or more inductive elements of the heating arrangement 104 to thereby generate the flow of aerosol.

The heating tube may have a circular cross-section or may have a cross-section of another shape. The adjustment mechanism may allow a dimension of the heating chamber 102 to be adjusted by adjusting a diameter of the heating tube. For example, a diameter of the heating tube may be adjustable to adjust the diameter of the heating chamber 102. Additionally, or alternatively, a length of the heating tube may be adjustable to adjust the length of the heating chamber.

It will be appreciated that the device 100 may comprise other components not shown in FIG. 1, such as ventilation inlets/outlets, a control interface, etc. It should be noted that FIG. 1 is merely a schematic sketch showing a number of components that may be included in the device 100. FIG. 1 is not intended to communicate particular positions of various components.

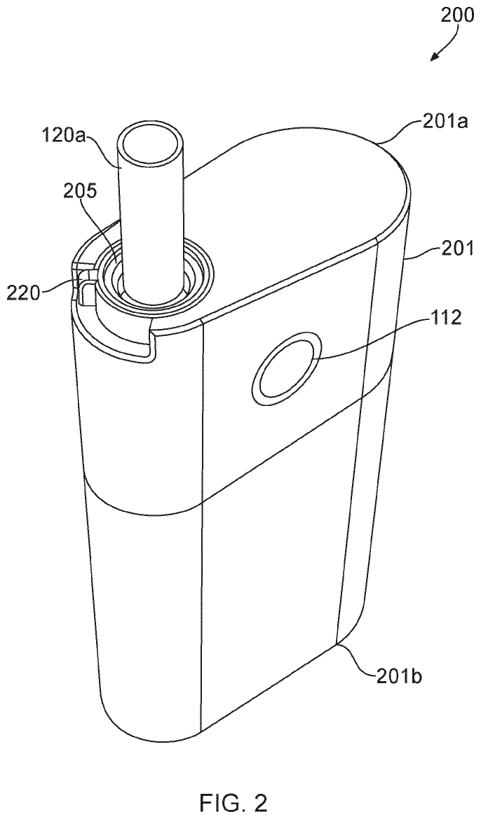

FIG. 2 shows a perspective view of a second device 200 according to an example of the disclosure. The second device 200 may comprise any of the features of the device 100 which have been described above with reference to FIG. 1 and discussion of these features will not be repeated here. Like figure references will be used to denote those features already described with reference to FIG. 1.

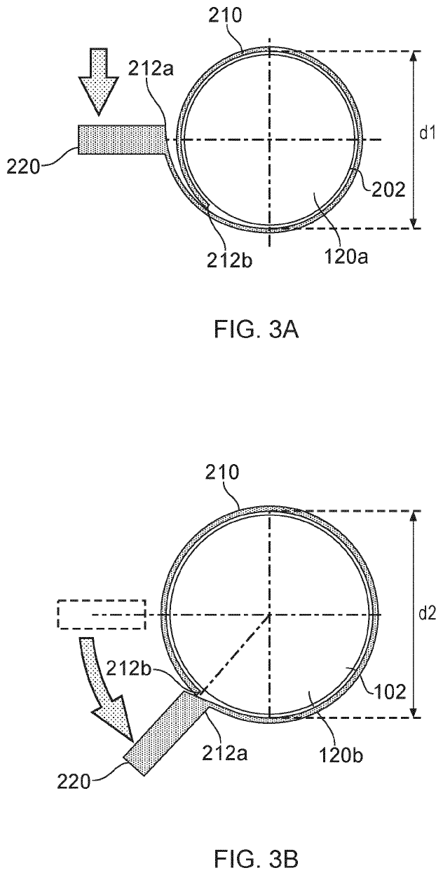

In FIG. 2, a consumable 120a is shown received in a heating chamber (202, FIG. 3A) inside a housing 201 of the device 200, to be heated. A portion of the consumable 120a protrudes from a proximal opening 205 and is arranged to be inhaled on by the user.

In the device 200, the heating chamber 202 is defined by a heating tube 210, which in this example is a susceptor tube. The susceptor tube 210 cannot be seen in FIG. 2, but a top-down schematic representation, viewed along an axial direction of the susceptor tube 210, is shown in FIG. 3A and FIG. 3B.

The susceptor tube 210 is formed of a sheet of material having opposing axial edges comprising a first edge 212a and a second edge 212b. The sheet of material is rolled into a cylindrical tube which defines the chamber 202. The axial edges 212a, 212b run axially, or longitudinally, from the proximal end 201a of the device 200 to the distal end 201b of the device 200.

A user can adjust the diameter of the heating chamber 202 of the device 200 by engaging a lever 220, which in this example connects to the edge 212a, to adjust a degree of overlap of the edges 212a, 212b of the susceptor tube 210. In this example, the lever 220 is configured to cause movement of the first edge 212a when the lever 220 is rotated about the axial direction, as indicated by the arrows in FIGS. 3A and 3B.

Rotation of the lever 220 about the axial direction allows the susceptor tube 210 to be configured between a first configuration, shown in FIG. 3A, wherein the susceptor tube 210 has a first internal diameter d1, and a second configuration, shown in FIG. 3B, wherein the susceptor tube 210 has a second internal diameter d2. In one example, the first internal diameter d1 is around 5.4 mm and the second internal diameter d2 is around 6.8 mm.

The second diameter d2 is greater than the first diameter d1. Accordingly, the user may adjust the diameter of the susceptor tube 210 to allow the heating chamber 102 to accommodate different consumables having different diameters. In the first configuration shown in FIG. 3A, the susceptor tube 210 is configured to accommodate the first consumable 120a. In the second configuration shown in FIG. 3B, the susceptor tube 210 is configured to accommodate a second consumable 120b having a diameter greater than the diameter of the first consumable 120a. In one example, the first consumable 120a has a diameter of around 5.40 mm while the second consumable 120b has a diameter of around 6.68 mm. In some examples, the first consumable 120a and the second consumable 120b have different lengths to one another. In one example, the first consumable 120a has a length of around 83 mm while the second consumable 120b has a length of around 75 mm.

Adjustment of the diameter of the susceptor tube 210 may allow the susceptor tube 210 to closely fit the diameter of a particular one of the consumables 120a, 120b. Thus, the user may use whichever of the consumables 120a, 120b they desire by configuring the susceptor 210 in the appropriate configuration.

As mentioned above, the lever 220 adjusts the diameter of the susceptor tube 210 by allowing the degree of overlap of edges 212a, 212b to be adjusted. In this example, in the first configuration the edges 212a, 212b overlap one another. In the second configuration the edges 212a, 212b are substantially aligned. That is, in the second configuration having the larger diameter, the edges 212a, 212b of the susceptor tube 210 may form a tube with substantially no gaps between the edges 212a, 212b, but without the edges 212a, 212b substantially overlapping one another, as shown in FIG. 3B. In some examples, the edge 212a may abut the opposing side of the susceptor tube 210, e.g. the opposing edge 212b or an interior wall of the susceptor tube 210. This may provide a conductive path around the circumference of the portion of the susceptor tube 210 which defines the heating chamber 202. In other examples, there may be a gap between the second edge 212b and the opposing side of the susceptor tube 210, such that no complete conductive path around the circumference is formed.

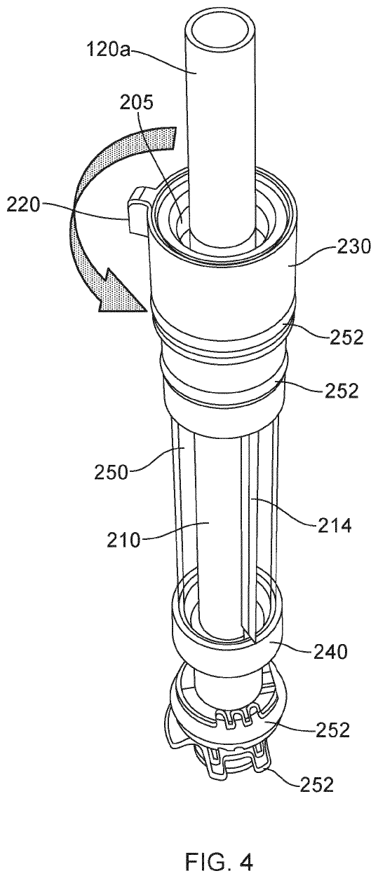

FIG. 4 shows a perspective view of the susceptor tube 210 and other additional internal components of the device 200 in isolation from the housing 201. The components shown in FIG. 4 form an assembly for accommodating the consumables 120a, 120b. FIG. 4 again shows the first configuration in which the narrower consumable 120a is received in the consumable and the susceptor tube 210 has the smaller first diameter d1. The assembly is shown in isolation to the housing 201 and other components of the device 200 for the purposes of clarity.

FIG. 4 shows a proximal component 230 of the device 200 which defines a proximal end of the heating chamber 202 and defines the opening 205 through which a consumable is inserted into the heating chamber 202. A distal component 240 defines a distal end of the heating chamber 102. The susceptor tube 210 is contained within an isolation tube 250, which is held in place between the proximal component 230 and the distal component 240. The isolation tube 250 may be generally tubular and at least partially surround the susceptor tube 210. The isolation tube 250 may be constructed from an insulating material, such as a plastics material for example. In this particular example, the isolation tube 250 is constructed from polyether ether ketone (PEEK). The isolation tube 250 may help insulate the various components of the device 200 from the heat generated in the susceptor 210. The isolation tube 250 may also be configured to prevent the egress of materials from the chamber 202 into portions of the device 200 exterior to the isolation tube 250.

One or more sealing rings 252 may encircle parts of the proximal component 230 and the distal component 240. The sealing rings 252 may be configured to seal the assembly shown in FIG. 4 in the housing 201 of the device 200, for example to provide an airtight seal in a recess of the device 200 in which the assembly is configured to be fitted.

The lever 220 is situated on the proximal component 230. The lever 220 is configured to cause rotation of the proximal component 230 within the housing 101. The proximal component 230 is rigidly attached to a first edge 212a of the susceptor tube 210, as described above, and thus when rotated causes the susceptor tube 210 to be configured between the first configuration and the second configuration. The susceptor tube 210 comprises a fin 214 which acts to anchor the susceptor tube 210 to the proximal component 230, the distal component 240 and the isolation tube 250.

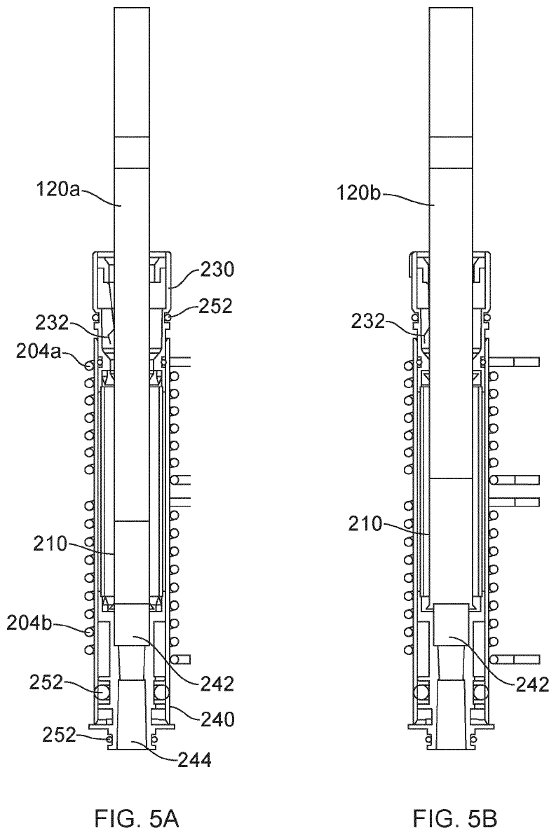

Turning now to FIGS. 5A and 5B, the assembly shown in FIG. 4 is shown in a cross-sectional representation, respectively in the first configuration and in the second configuration. FIG. 5A shows the susceptor tube 210 in the first, narrower, configuration and accommodating the first consumable 120a. FIG. 5B shows the susceptor tube 210 in the second, wider, configuration and accommodating the second consumable 120b.

Shown in FIGS. 5A and 5B are further features of the device 200 including a pair of inductor coils 204a, 204b which surround the heating chamber 202 and the susceptor tube 210. In examples, devices according to the disclosure which use induction heating may comprise one coil, or any other number of coils. Use of more than one coil may allow zones of the susceptor tube 210 to be independently heated. For example, the inductor coils 204a, 204b may be independently controllable to heat the respective zones of the susceptor tube 210 which they surround.

The distal component 240 defines a stop 242 at the distal end of the heating chamber 202 which is configured to abut the distal end of whichever of the consumables 120a, 120b is received in the chamber 202. Although not visible in the figures, the stop 242 may comprise a stepped arrangement which allows the thinner first consumable 120a to be inserted into the chamber 202 to a greater depth than the thicker second consumable 120b. For example, the stop 242 may comprise an aperture having a diameter which allows the first consumable 120a to be inserted through while preventing the second consumable being inserted through. As mentioned above, in this example, the first consumable 120a is longer than the second consumable 120b. The different depths to which the consumables 120a, 120b are permitted to be inserted into the chamber 202 by the stop 242 may act to make the length of each consumable 120a, 120b which protrudes from the opening 205 substantially equal. For example, in some examples, a filter portion and various other components of the consumables 120a, 120b may be arranged at the same distance from the respective proximal ends of the consumables 120a, 120b. Thus, having the consumables protrude from the opening 205 by substantially equal amounts may allow ventilation holes and the like to be positioned in the same axial location with respect to the opening 205.

The distal component 240 may further comprise a cleaning tube 244 which may be accessible from a distal end of the housing 201 to allow cleaning of the interior of the chamber 202.

Inside of the proximal component 230 is a flexible retention element 232. The flexible retention element 232 is configured to engage whichever of the first consumable 120a and the second consumable 120b is received in the chamber 202 near the proximal opening 205 to hold the consumable in position.

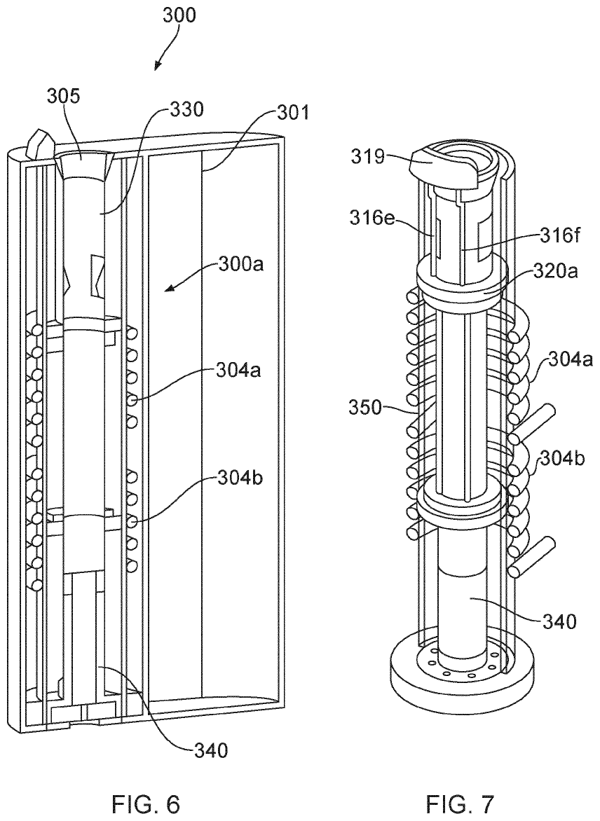

FIG. 6 shows a third device 300 according to the disclosure. The device 300 may share features described for previous embodiments, which are shown with like figure references and a description of which will not be repeated.

The device 300, similarly to as described with reference to the device 200, comprises a proximal component 330 which forms a proximal tubular section having an opening 305 which allows a consumable 120a, 120b to be inserted to be received in a heating chamber 302 defined by a susceptor tube 310. The proximal component 330 is tube of fixed diameter, and may, for example, be formed of a plastics material, such as PEEK. The device 300 similarly comprises a distal portion 340 and an isolation tube 350 which may comprise any of the features described with reference to the second device 200. As in the second device 200, first and second induction coils 304a, 304b surround the susceptor tube 310.

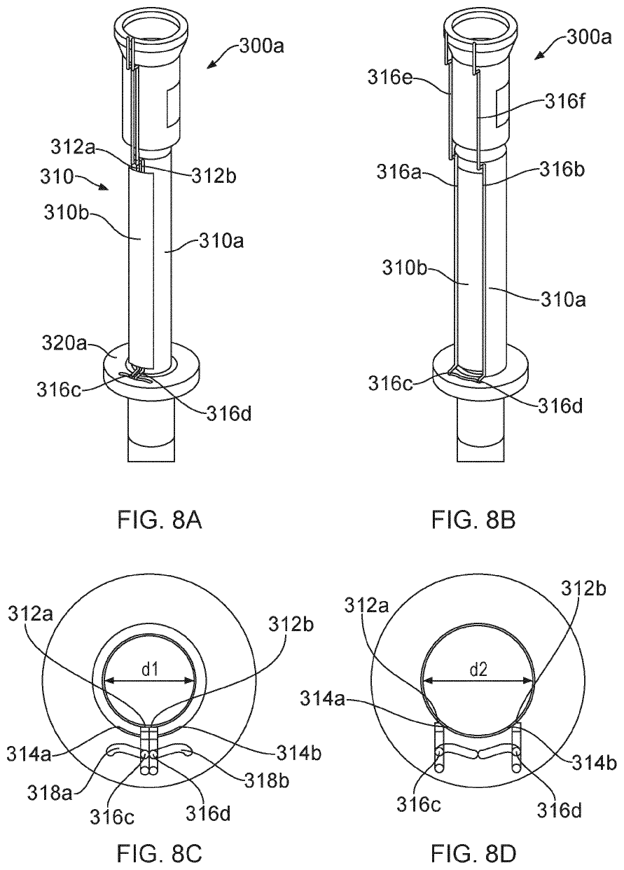

FIG. 7 shows the susceptor tube 310 and other internal components of the device 300 in isolation from a housing 301 of the device 300. A diameter of the susceptor tube 310 is adjustable, as will now be described with reference to FIGS. 8A to 8D.

FIGS. 8A and 8B show, each in a perspective view, the susceptor tube 310 and other components of the device 300 which form an assembly for accommodating each of the consumables 120a, 120b. The susceptor tube 310 of the device 300 is shown in a first configuration in FIG. 8A, and in a second configuration in FIG. 8B. FIGS. 8C and 8D, respectively, illustrate aspects of the first configuration and second configuration in a top-down schematic view.

In this example, the susceptor tube 310 is formed of a first part 310a and a second part 310b. In a similar manner to the susceptor tube 210 of device 200, the first part 310a of the susceptor tube 310 comprises opposing edges 312a, 312b whose position relative to one another is adjustable to adjust the diameter of the susceptor tube 310.

In the first configuration, shown in FIGS. 8A and 8C, the edges 312a, 312b substantially abut one another to form a closed tube having the first diameter d1 for accommodating the first consumable 120a. In the second configuration, shown in FIGS. 8B and 8D, the edges 312a, 312b are separated from one another and do not overlap one another. In this configuration, the susceptor tube 310 takes the larger second diameter d2 which is suitable for accommodating the second consumable 120b. In the second configuration, the second part 310b is situated between the edges 312a, 312b and forms a section of the circumference of the susceptor tube 310.

In examples, the second part 310b of the susceptor tube 310 is formed of susceptor material, such that the circumference of the susceptor tube 310 in the second configuration remains a closed loop of susceptor material. In the first configuration, however, it can be seen that the second part 310b does not form a part of the closed loop which defines the chamber 302 and is in fact located outside of the closed loop which defines the chamber 302.

An adjustment mechanism allows a user to configure the susceptor tube 310 between the first configuration and the second configuration. The adjustment mechanism acts to move the edges 312a, 312b of the first part 310a of the susceptor tube 310 between their respective positions in the first configuration, wherein they abut one another, and their respective positions in the second configuration, wherein the edges 312a, 312b abut respective outer axial edges 314a, 314b of the second part 310b of the susceptor tube 310.

The adjustment mechanism for repositioning the edges 312a, 312b of the first part 310a of the susceptor tube 310 comprises a pair of guide elements 316a, 316b. A first guide element 316a is attached to the first part 310a along the first edge 312a. A second guide element 316b is attached to the first part 310a along the second edge 312b. Each of the guide elements 316a, 316b may be comprise elongate member which runs along a respective one of the edges 312a, 312b. Each of the guide elements 316a, 316b has a distal portion 316c, 316d for interacting with a respective guide slot 318a, 318b. Each of the distal portions 316c, 316d is radially separated from but connected to an elongate portion of the guide element 316a, 316b which runs along a respective one of the edges 312a, 312b. Thus, movement of the distal portions 316c, 316d along the guide slots 318a, 318b causes movement of the edges 312a, 312b and reconfiguring of the susceptor tube 310.

The guide slots 318a, 318b are formed in a distal annular member 320a at the distal end of the susceptor tube 310. A first guide slot 318a defines a track in which the first distal portion 316c of the first guide element 316a moves. A second guide slot 318b similarly defines a track for the second distal portion 316d of the second guide element 316b. In the first configuration, as shown in FIG. 8C, the distal portions 316c, 316d are adjacent one another at one end of their respective tracks 318a, 318b while in the second configuration, shown in FIG. 8D, the distal portions 316c, 316d are at an opposite end of the tracks 318a, 318b and are at a furthest point from one another allowed by the tracks 318a, 318b.

Respective proximal portions 316e, 316f of the guide elements 316a, 316b are located towards the opening 305 to the chamber 302. The proximal portions 316e, 316f are movable by a user by use of a knob 319, best seen in FIG. 7. Movement of the knob 319 causes the guide elements 316a, 316b to move along the tracks 318a-318d. Movement of the proximal portions 316e, 316f therefore causes reconfiguration of the susceptor tube 310 between the first configuration and the second configuration.

A proximal annular member 320b, shown in FIG. 7, similarly to the distal annular member 320a may comprise guide slots to guide respective portions of the guide elements towards the proximal portions 316e, 316f, in the same manner as the guide slots 318a, 318b guide the distal portions 316c, 316d.

The knob 319 engages the proximal portions 316e, 316f to move said portions while allowing the distance between said portions to change to configure the susceptor tube 310 between the first configuration and the second configuration. In moving between the first configuration and the second configuration, the guide elements 318a, 318b move along the tracks 318a, 318b and the tracks in the annular proximal member 320b. In an example, the knob 319 is movable inwardly and outwardly along a radial direction of the chamber 302. In this example, moving the knob 319 radially inwards forces the guide elements 316a, 316b together to configure the susceptor tube 310 in the first (smaller diameter) configuration and moving the knob 319 radially outwards moves the guide elements 316a, 316b apart and configures the susceptor tube 310 in the second (larger diameter) configuration.

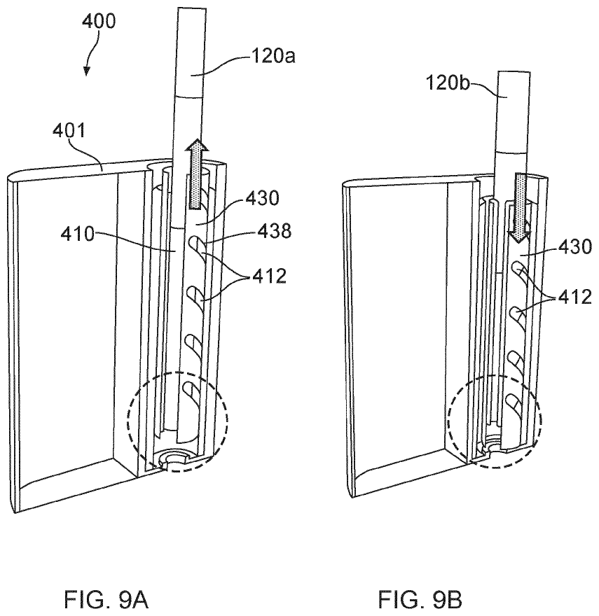

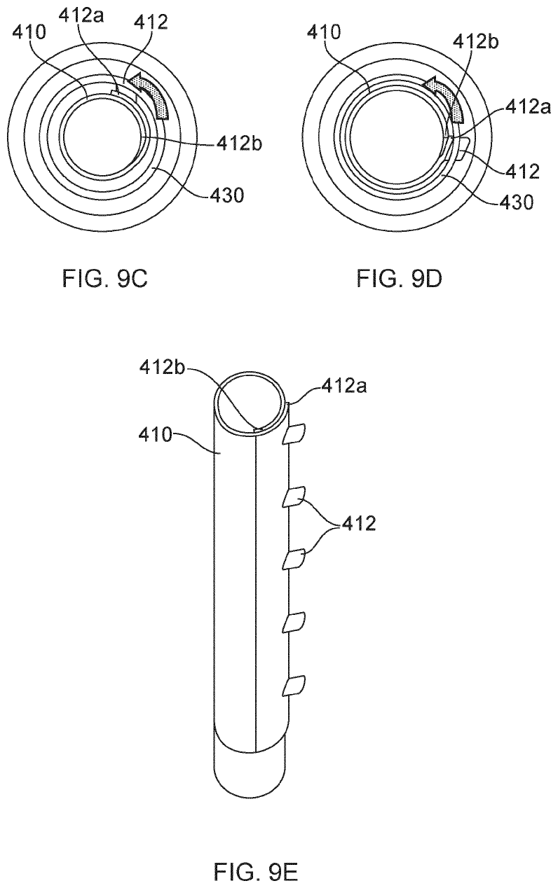

FIGS. 9A and 9B show a fourth device 400 according to the disclosure. The fourth device 400 is similar to the second device 200 and the third device 300 in that it comprises a heating tube 410 whose diameter is adjustable to adjust the diameter of a heating chamber 402. In the fourth device 400, the heating tube 410 is a flexible resistive heating tube, rather than a susceptor. Nevertheless, the principles which allow the dimensions of the heating chamber 402 of the fourth device 400 to be adjusted may be applied in an inductive heating device.

The fourth device 400 comprises a frame 430 which surrounds the heating tube 410. The heating tube 410 comprises a set of guide elements 412 on its outer surface, which are best seen in FIG. 9E. The frame 430 comprises a set of guide tracks 438. In the example shown in figures the heating tube 410 comprises five guide elements 412 for interacting respectively with five guide tracks 438 of the frame 430. However, it will be appreciated that any number of guide elements 412 and guide tracks 438 may be used in other examples.

FIG. 9A shows the device 400 in the first configuration wherein the susceptor tube 410 takes its minimum diameter and wherein the first consumable 120a is received by the heating tube 410.

FIG. 9B shows the device 400 in the second configuration wherein the susceptor tube 410 takes its maximum diameter and wherein the second consumable 120a is received by the heating tube 410.

FIGS. 9C and 9D show the frame 430 and heating tube 410 in the first and second configurations respectively, in a top-down view along the axial direction of the device 400. FIG. 9E shows the heating tube 410 in isolation in a perspective top-down view.

The frame 430 is movable in an axial direction, as illustrated by the arrows in FIGS. 9A and 9B. As the frame 430 moves along the axial direction, the guide elements 412 are guided along the guide tracks 438. The guide elements 412 are rigidly attached along a first edge 412a of the sheet which forms the heating tube 410. The first edge 412a is caused to move under the influence of the guide elements 412 being guided by the guides 438. An opposing axial, inner, edge 412b of the sheet which forms the heating tube 410 is held in place. Accordingly, as the frame 430 moves and the guide elements 412 are guided along the tracks 438 the degree of overlap of the edges of the heating tube 410 is adjusted, thereby adjusting the diameter of the heating tube 410.

In FIGS. 9A and 9B, the guides 438 can be seen to each define a track along a section of a respective helix. In this example, the guides 438 all define tracks along sections of respective left-handed helices. The heating tube 410 remains in a fixed position with respect to the axial direction while the frame 430 moves in the axial direction. Accordingly, as the frame 430 is moved downwardly along the axial direction, i.e. towards the distal end of the device 400, the guide elements 412 are forced to move along the guides 438, in a clockwise direction when viewed from the proximal end of the device 400. This means that as the frame 430 is moved downwardly, the diameter of the heating tube 410 is caused to increase.

In some examples, the heating tube 410 is biased towards the first configuration having the smaller diameter. For example, the heating tube 410 may be biased in such a way that it tends to constrict itself towards the first configuration. Thus, moving the frame 430 downwardly may comprise working against the bias of the heating tube 410. This biasing action may provide for the heating tube 410 to form a tight fit around whichever of the first consumable 120a and the second consumable 120b is inserted. For example, the user may actuate movement of the frame 430 downwardly, either by pressing the frame 430 or by actuating a button or portion of the housing 401 which is configured to cause the frame 430 to move. While the user holds the frame 430 downwardly to configure the device 400 in the second configuration the user may insert either of the first consumable 120a or the second consumable 120b into the heating tube 410. Once the first consumable 120a or the second consumable 120b is inserted, the user may release the frame 430 from the downward position. The heating tube 410 then may act under the bias to contract and to form a tight fit around the inserted consumable. It will be appreciated that in some examples in the second configuration when the frame 430 is fully pressed down the diameter of the heating tube 410 may be slightly larger than the diameter of the second consumable 120b to allow insertion of the second consumable 120b. In such examples, upon release of the frame 430 the heating tube 410 may contract slightly to fit the diameter of the second consumable 120b. To remove a consumable from the heating tube 410, the user may again push down on the frame 430 to increase the diameter of the heating tube 410 and loosen any fit with the inserted consumable to allow the consumable to be pulled free.

Examples which are biased to return to a smaller diameter, as described above, may allow the heating tube 410 to closely fit the diameter of an inserted consumable. This may provide for effective heating of the consumable. Further, rather than manually configuring the device in a specific predetermined configuration intended for use with either the first consumable 120a or the second consumable 120b, the user may simply press the frame 430 downwardly, insert the consumable and then release the frame 430 which may automatically form a close fit to the inserted consumable.

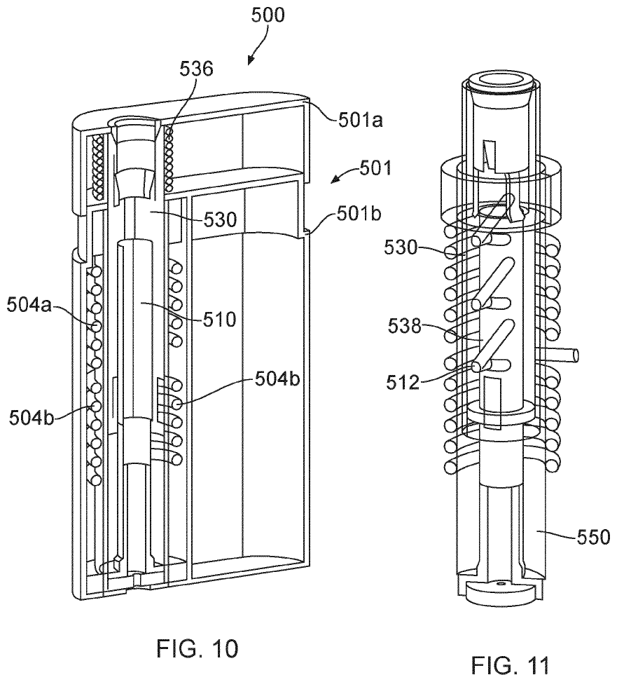

A fifth device 500 according to the disclosure is shown in FIG. 10. In a similar manner to previous examples, an assembly comprising a heating tube 510 and other components in isolation to the remainder of the device 500 is shown in FIG. 11.

Similarly to the fourth device 400, the heating tube 510 of the fifth device 500 comprises a set of guide elements 512. The fifth device 500 also comprises a frame 530 comprising a set of guides 538 which surrounds the heating tube 510. Axial movement of the frame 530 controls circumferential movement of the guide elements 512 to thereby adjust the diameter of the heating tube 510 by adjusting a degree of overlap of the edges of the heating tube 510. The diameter of the heating tube 510 is thereby adjustable by moving the frame 530 in the axial direction, in a similar manner as to described for the fourth device 400.

FIG. 11 shows the heating tube 510 in a first configuration having the first, minimum, diameter. As with the fourth device 400, pushing the frame 530 of the fifth device 500 downwardly configures the heating tube 510 from the first configuration shown in FIG. 11 to the second configuration (not shown).

In this example, the guides 538 run along sections of respective right-handed helices. Thus, when the frame 530 is moved downwardly, the guides 538 cause the guide elements 512 to move to the right, which, due to the way that the heating tube 510 is wrapped and the position of the guide elements on the outer surface of the heating tube 510, causes the diameter of the heating tube 510 to be increased.

In the fourth device 400 and the fifth device 500, the guide elements may be located on the outer overlapped edge of the sheet which forms the susceptor tube 410, 510, as in shown in FIG. 9C. However, it may also be the case that the guide elements are located on an inner overlapped edge of the sheet which forms the susceptor tube 410, 510. In such examples, the outer overlapped edge of the sheet may comprise a respective guide slot to allow each of the guide elements on the inner overlapped edge to protrude through the outer overlapped edge to communicate with the guides 438, 538 in the frame 430, 530. This may contribute to allowing the heating tube to keep its tubular shape as its diameter is adjusted.

As shown in FIG. 10, the fifth device 500 comprises a two-part housing 501 comprising a top section 501a and a bottom section 501b. The top section 501a is movable in the axial direction with respect to the bottom section 501b. Pressing down on the top section 501a moves the frame 530 downwardly and thereby increases the diameter of the heating tube 510 to allow insertion of a consumable 120a, 120b. A biasing element 536, in this example a coiled spring, is positioned inside the top section 501a to bias the top section 501a away from the lower section 501b. Once the user stops pressing down on the top section 501a, the top section 501a moves upwardly under the action of the biasing element 536 to cause the frame 530 to move upwardly and to contract the heating tube 510 to fit the diameter of the inserted consumable.

The fifth device 500 is an induction heating device and the heating tube 510 is a susceptor tube. As in previously described examples of induction heating devices, inductor coils 504a, 504b surround the susceptor tube 510 and frame 530, and the susceptor tube 510 and frame 530 are contained within an isolation tube 550.

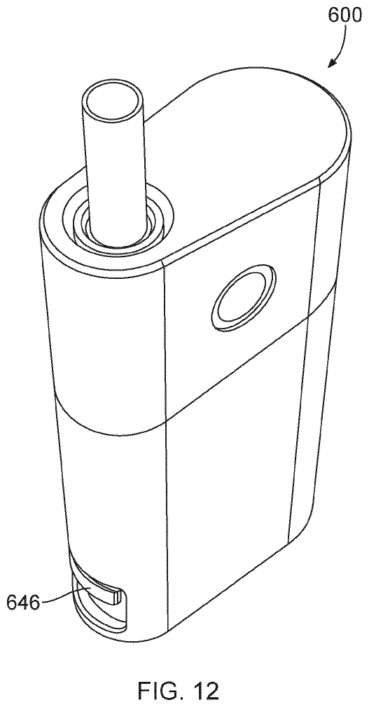

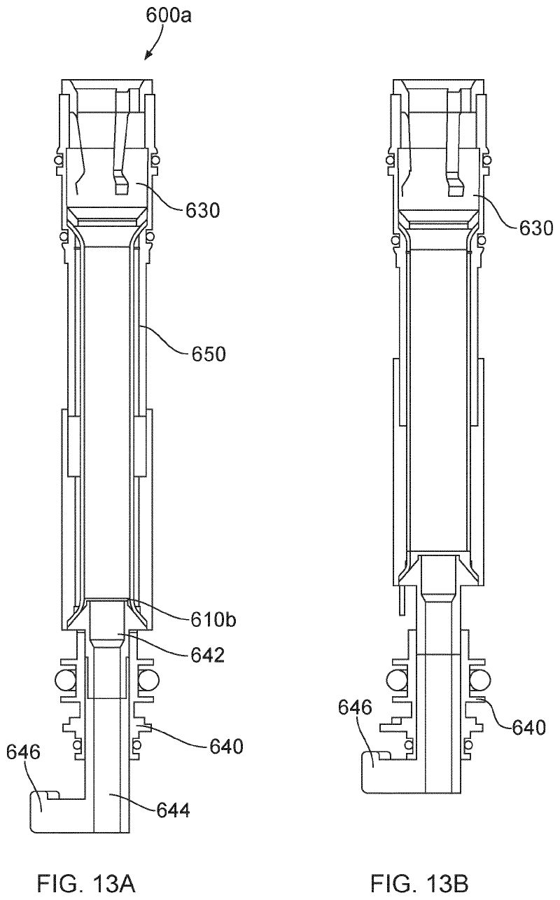

A sixth device 600 according to the disclosure is shown in FIG. 12 and in FIGS. 13A and 13B. FIG. 12 shows a perspective view of the sixth device 600. The sixth device 600 shares features of previously described devices which will not be repeated.

FIG. 13A and FIG. 13B show cross-sectional views of an assembly of the device 600 comprising a heating tube 610 and other components described above, such as a proximal component 630 to which a proximal end of the heating tube 610 is attached.

In the device 600 the heating tube 610 is a stretchable heating tube. That is, the heating tube 610 is configured such that when its length is increased its diameter is decreased. Conversely, when the length of the heating tube 610 is decreased its diameter is increased. The heating tube 610 may be formed with a braided structure. In examples, the heating tube 610 is a susceptor tube which comprises a susceptor material, such as steel. A proximal end 610a of the heating tube 610 is attached to the proximal component 630 and held in a fixed position. A distal end 610b of the heating tube 610 is attached to a distal component 640 which is moveable in the axial direction to configure the length and diameter of the heating tube 610. The proximal and distal ends 610a, 610b of the heating tube 610 may flare outwardly towards the respective ends of the tube 610. The distal component 640 comprises a stop 642 which a distal end of a consumable received in the heating tube 610 is configured to abut. A distal tube 644 allows for draining of condensate and the like from the distal end of the device 600 and an opening in the distal end of the device housing may allow a user to access the interior of the distal tube 644, for example, for cleaning purposes.

The distal component 640 is moveable in the axial direction by means of a switch 646 which is attached to the distal tube 644 and is configured to extend transversely to the axial direction. The switch 646 is presented to the user and is moveable in a recess in the outer face of the housing of the device 600, as shown in FIG. 12.

The switch 646 can be moved between a downward position (FIG. 13A) and an upward position (FIG. 13B) to configure the heating tube 610 between the first configuration for receiving the first consumable 120a and the second configuration for receiving the second consumable 120b. Moving the switch 646 moves the distal component 640 including the stop 642 and thus reconfigures the length of the heating tube 610 for receiving consumables of different lengths and of different diameters. As in other induction heating devices described herein, the susceptor tube 610 may be contained in an isolation tube 650 and one or more induction coils (not shown) may surround the isolation tube to generate the varying magnetic field for heating the susceptor tube 610.

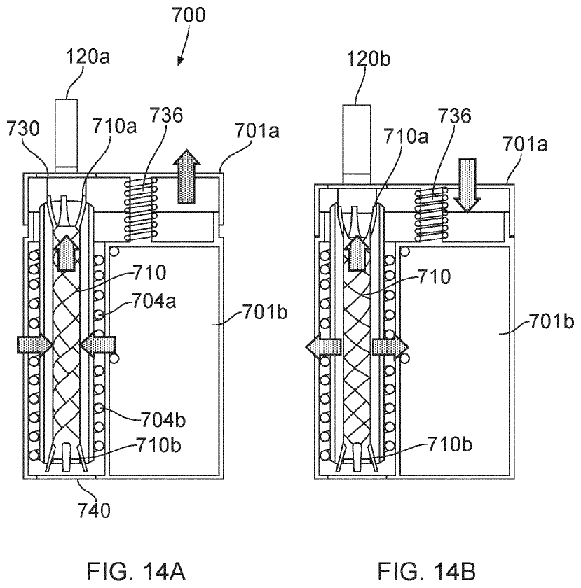

A seventh device 700 according to an example of the disclosure is shown in a schematic representation in FIG. 14A and FIG. 14B. The seventh device 700 shares features described with reference to earlier examples which will not be repeated. The seventh device 700 employs a similar stretchable heating tube 710 to the sixth device 600. In this example, the heating tube 710 is formed of steel. A proximal end of the heating tube 710 is attached to a proximal component 730 of the device 700 via flexible strings 710a. A distal end of the heating tube 710 is attached to a distal component 740 of the device 700 via flexible strings 710b. The seventh device 700 comprises a similar biasing mechanism to that described for the fifth device 500. That is, a top portion 701a of a housing of the seventh device 700 is biased away from a lower portion 701b of the housing by a biasing element 736. In this example, again, the biasing element 736 is a coiled spring located inside the top portion 701a and configured to bias the top portion 701a away from the lower portion 701b. Any other suitable biasing element may be used in other examples. The user may, accordingly, depress the top portion 701a to decrease the length and increase the diameter of the heating tube 710 to allow insertion of either the first consumable 120a or the second consumable 120b. The connection of the flexible strings 710a, 710b to the heating tube 710 allows the heating tube 710 to deform evenly when the housing portion 701a is depressed and released.

Once a consumable 120a, 120b is inserted, the user releases the top portion 701a of the device 700 to allow the biasing action to cause the top portion 701a to move upwardly to stretch the heating tube 710 to cause the heating tube 710 to constrict to form a tight fit around the inserted consumable 120a, 120b. FIG. 14A shows the device 700 with the first consumable 120a inserted into the heating tube 710. FIG. 14B shows the device 700 with the second consumable 120b inserted into the heating tube 710. In this example, the seventh device 700 is an inductive heating device and the heating tube 710 is a susceptor tube.

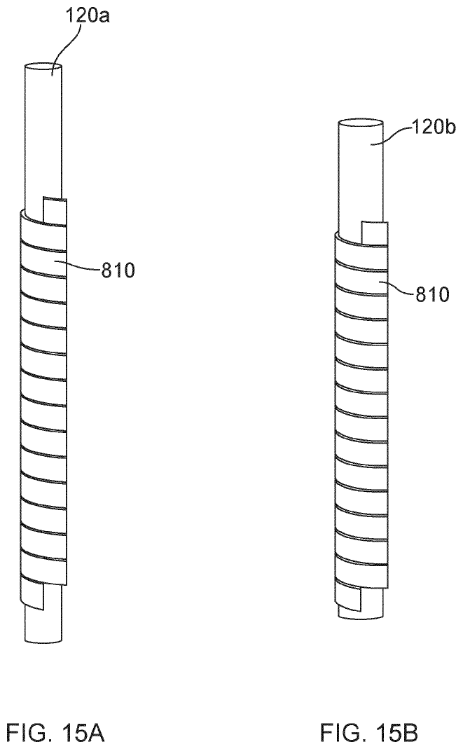

In another example, shown in FIG. 15A and FIG. 15B, a helical coil 810 defines a stretchable tube for accommodating differently-dimensioned consumables in a device similar to examples described above. Increasing a length of the coil 810 causes a decrease in a diameter of the coil 810 while, conversely, decreasing the length of the coil causes an increase in the diameter of the coil. Similarly, increasing the diameter of the coil 810 may cause the length of the coil 810 to decrease while decreasing the diameter of the coil 810 may cause the length of the coil 810 to increase. The coil 810 is thereby able to fit to a plurality of differently sized consumables, e.g. the first consumable 120a and the second consumable 120b, as is shown in FIG. 15a and FIG. 15b respectively. The coil 810 may be configured such that a pressure exerted by the coil 810 on a consumable accommodated by the coil 810 is substantially constant over the length of the coil. In some examples, the coil 810 may be formed of a low mechanical resistance polymer, or of card. For example, the coil 810 may be formed of a ceramic elastomer or the like. In another example the coil 810 may be formed of PTFE, e.g. the coil 810 may comprise a Teflon wrap.

The above embodiments are to be understood as illustrative examples of the disclosure. Further embodiments of the disclosure are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

1. A non-combustible aerosol provision device for generating an aerosol from an aerosol-generating material, the non-combustible aerosol provision device comprising:
a chamber for receiving a consumable comprising aerosol-generating material to enable the non-combustible aerosol provision device to generate an aerosol from the aerosol-generating material; and
an adjustment mechanism to allow a user to adjust a dimension of the chamber to allow the chamber to accommodate, one at a time, each of a plurality of consumables having different sizes.

2. The non-combustible aerosol provision device according to claim 1, further comprising a tube, wherein an interior of the tube defines the chamber, and wherein the adjustment mechanism allows adjustment of a dimension of the tube to thereby adjust the dimension of the chamber.

3. The non-combustible aerosol provision device according to claim 2, wherein a sheet of material is arranged to form the tube, the sheet of material comprising two respective opposing longitudinal edges, and wherein the adjustment mechanism allows a position of the opposing longitudinal edges of the sheet of material with respect to one another to be adjusted to adjust a diameter of the tube.

4. The non-combustible aerosol provision device according to claim 3, wherein the opposing longitudinal edges of the sheet of material are configured, in at least some configurations in use, to overlap one another, and wherein the adjustment mechanism allows a degree of overlap of the opposing longitudinal edges to be adjusted to adjust the diameter of the tube.

5. The non-combustible aerosol provision device according to claim 3, wherein the opposing longitudinal edges of the sheet of material are configured such that, in at least some configurations, the opposing longitudinal edges do not overlap one another, and wherein a distance between the opposing longitudinal edges with respect to a circumferential direction of the tube is adjustable by the adjustment mechanism.

6. The non-combustible aerosol provision device according to claim 5, wherein the tube further comprises a second portion, wherein the second portion is configured to form a portion of the tube between the opposing longitudinal edges when the opposing longitudinal edges of the sheet of material are not overlapping one another.

7. The non-combustible aerosol provision device according to claim 2, wherein the adjustment mechanism comprises a frame having one or more guides and the tube comprises one or more guide elements, each of the one or more guide elements being configured to engage with a respective one of the one or more guides to cause a diameter of the tube to be adjusted when the frame is moved relative to the tube.

8. (canceled)

9. The non-combustible aerosol provision device according to claim 2, wherein the adjustment mechanism is configured to adjust a length of the tube.

10. The non-combustible aerosol provision device according to claim 9, wherein adjustment of one of a diameter of the tube or the length of the tube causes adjustment of the other of the diameter of the tube or the length of the tube.

11. The non-combustible aerosol provision device according to claim 10, wherein a reduction in one of the length of the tube or the diameter of the tube causes an increase in the other of the length of the tube or the diameter of the tube, and an increase in one of the length of the tube or the diameter of the tube causes a decrease in the other of the length of the tube or the diameter of the tube.

12. The non-combustible aerosol provision device according to claim 9, wherein the tube comprises one of an expandable braided structure or a helical coiled structure.

13. (canceled)

14. The non-combustible aerosol provision device according to claim 11, wherein the adjustment mechanism comprises a biasing element arranged to bias the length of the tube to a maximum length and wherein the adjustment mechanism allows the user to work against the biasing element to decrease the length of the tube and thereby to increase a diameter of the chamber.

15. The non-combustible aerosol provision device according to claim 1, wherein the adjustment mechanism comprises a biasing element configured to act to decrease the dimension of the chamber, and wherein the adjustment mechanism allows the user to work against the biasing element to increase the dimension of the chamber to allow insertion of the consumable comprising aerosol-generating material.

16. The non-combustible aerosol provision device according to claim 1, wherein the adjustment mechanism allows the user to configure the chamber between a first configuration in which the chamber has a first width and a second configuration in which the chamber has a second width different from the first width.

17. The non-combustible aerosol provision device according to claim 1, wherein the adjustment mechanism comprises an actuator engageable by the user to operate the adjustment mechanism to adjust the dimension of the chamber.

18. The non-combustible aerosol provision device according to claim 17, wherein the actuator either comprises a lever which is rotatable in a circumferential direction with respect to the chamber to operate the adjustment mechanism, or is moveable in a longitudinal direction with respect to the chamber to operate the adjustment mechanism.

19. (canceled)

20. The non-combustible aerosol provision device according to claim 17, wherein the actuator allows the user to work against a biasing element to increase a diameter of the chamber to allow insertion of a consumable and wherein the adjustment mechanism is configured, when a button is released, to cause contracting of the chamber to fit the consumable.

21. The non-combustible aerosol provision device according to claim 17, wherein the actuator comprises a portion of an outer housing of the non-combustible aerosol provision device which is moveable relative to the chamber.

22. (canceled)

23. The non-combustible aerosol provision device according to claim 1, wherein the adjustment mechanism is configured to adjust a length of the chamber.

24. The non-combustible aerosol provision device according to claim 2, wherein the tube is one of:
a susceptor element configured to be inductively heated by an inductive element of the non-combustible aerosol provision device to thereby heat the aerosol-generating material received in the chamber; or
configured to be heated by a resistive heater to thereby heat the aerosol-generating material received in the chamber.

25. (canceled)

26. A non-combustible aerosol provision device according to claim 1, wherein the non-combustible aerosol provision device is a tobacco heating product configured to receive and generate the aerosol from each of a plurality of consumables comprising tobacco and having different sizes.

27. A non-combustible aerosol provision device for generating an aerosol from an aerosol-generating material, the non-combustible aerosol provision device comprising:
a chamber for receiving a consumable comprising aerosol-generating material to enable the non-combustible aerosol provision device to generate an aerosol from the aerosol-generating material; and
an adjustment mechanism configured:
to allow a user to increase a width of the chamber to allow the consumable to be inserted into the chamber, and
once the consumable is inserted into the chamber, to decrease the width of the chamber to cause the chamber to fit to a width of the consumable.

28. A non-combustible aerosol provision system comprising the non-combustible aerosol provision device according to claim 1 and at least one consumable containing aerosol-generating material, the consumable being configured to be received by the non-combustible aerosol provision device to allow the non-combustible aerosol provision device to generate the aerosol from the aerosol-generating material.
